(19) United States
(12) Patent Application Publication
(10) Pub. No.: US 2020/0155045 A1
(43) Pub. Date: May 21, 2020

(54) System and Method for Changing a State of a Device

(71) Applicant: SPD Swiss Precision Diagnostics GmbH, Geneva (CH)

(72) Inventor: Paul Sharrock, Geneva (CH)

(21) Appl. No.: 16/636,288

(22) PCT Filed: Aug. 9, 2018
(86) PCT No.: PCT/EP2018/071630

§ 371 Date: Feb. 3, 2020

(30) Foreign Application Priority Data
Aug. 10, 2017 (GB) 1712811.7

(51) Int. Cl. A61B 5/145; A61B 10/00; A61B 5/00

(52) U.S. Cl. CPC A61B 5/14507; A61B 10/0012; A61B 10/007; A61B 5/1455; A61B 5/742; A61B 2562/0295; A61B 5/14546

(57) ABSTRACT

A system for changing a state of a reading device, the system comprising: a reading device arranged to operate in a first state for reading a result of a test performed using a test stick, the reading device comprising a detection means arranged to detect a first reading influenced by a detection region of the test stick; an inserter for changing a state of the reading device; wherein, when the inserter is inserted into the reading device, the detection means detects a second reading distinct from the first reading, the reading device being arranged to operate in a second state in response to detecting the second reading.

### FIELD

The disclosure relates to a system and method for changing a state of a device. In particular, the invention relates to a system and method for changing a state of an assay result reading device.

### BACKGROUND

Devices for the determination of analytes present in a sample, such as urine, are widely available over the counter and are common in professional use. Such devices are designed to be simple to use and, for example, provide information relating to ovulation, pregnancy and menopause. Currently available ovulation test devices include those that are intended for home use and are designed to be used by women who are either trying to become pregnant, or are deliberately avoiding pregnancy. Such products may provide an indication of a woman's fertility throughout the course of the woman's menstrual cycle, or indicate a woman's fertility during selected times of her menstrual cycle. Typically, these test devices comprise a specific area(s) where biochemical reactions with the analyte(s) of interest take place, thereby allowing the identification and/or quantification of the analyte(s) present in the sample from which the test result is determined. In certain ‘visual test devices’, the user may examine the specific area(s) and interpret the test result themselves, however in other ‘digital test devices’ a detection means may be used to interpret the specific area(s) and output the test result on a display located on or within the test device. Digital test devices can incorporate a power source (battery) and electronic circuitry to drive the detection means as well as a display and are typically designed to be disposable, having a defined battery capacity which has to be carefully balanced to ensure adequate longevity of the reading device. Ovulation tests therefore identify those days in a woman's cycle on which intercourse is most likely to lead to conception.

One known product defines three phases of fertility through urine hormone measurement. These phases of fertility may be termed “low” (lower chance of conceiving), “high” (increased chance of conceiving), which is determined by detecting a rise in the level of Estrone-3-Glucuronide (E3G), and “peak” (higher chance of conceiving), which provides an early warning of impending ovulation through detection of a surge in luteinising hormone (LH). Such a surge typically precedes ovulation by 24-36 hours. The results, in terms of low, high or peak, are displayed on a display of the digital ovulation test device to provide such information to a user.

The determination of ovulation typically requires testing to be performed on a daily basis, the measurements and results from previous days' testing being used in an algorithm to define the fertility state on the next testing occasion. The detection of hormones may be made through the combination of the ovulation test device (which acts as a reader) and a number of disposable test sticks, the test sticks typically incorporating the specific area(s) where biochemical reactions with the analyte(s) take place. The specific area(s) where biochemical reactions take place may be encompassed within a detection region. The specific area(s) may take the form of a line(s) known as a test line(s) or assay test line(s).

To perform a test, the user inserts a test stick into the reader and then applies a sample, which is usually urine. The user may apply the sample to the test stick as a first step and then insert the test stick into the reader. Alternatively, the test stick may already be placed into the reader before the application of a sample. The application of the sample to a dry (unused device, or unused test stick) is also known as running the device, the device becoming wet in the process and is subsequently referred to as a used or run device, (run test stick). In the instance where the hormones LH and E3G are being measured, the test stick incorporates two immunochromatographic assays that develop test lines on the test stick, the intensity of which is relative to the concentration of each analyte in the sample. The reader interprets the intensity of the test lines by virtue of the detection means, for example by illuminating the test stick and detecting a reflection from the test stick. Alternatively, a transmission of light through the test stick may be used. Values based on the changes in reflection or transmission of light due to the intensity of the assay lines and values derived from previous tests may be applied to an algorithm to determine a state of fertility. This result is passed to the user in the form of a qualitative result displayed on a display of the device. The result reflects the intended use of the product, i.e. to determine a fertility state.

Since test devices are often designed to be disposable after a defined period of use, any improvement to the functionality of the test device is heavily constrained by cost. Although device functionality may be improved by adding hardware, this would have a cost implication and may be undesirable in certain situations. As a result, known devices are restricted in their functionality due to the need to minimise cost. This often means that multiple different devices are required to satisfy different needs or perform different functions.

An example functionality that is desirable in test devices can be found in certain devices which also have wireless data connectivity in order to send ovulation, fertility or other data to an external device such as a mobile phone or a computer. Such a device may be a digital ovulation test device. It is imperative to balance the size and cost of the battery with the functions required by the test device. The size, cost and capacity of the battery in digital ovulation test devices hence presents a challenge to the manufacturer who would prefer to limit battery use and hence save battery by not continuously or excessively transmitting or receiving data wirelessly. Further, it may be undesirable to transmit data for an extended period as this provides a larger window of time for the data to be intercepted up by an unwanted device. Whilst the manufacturer of such devices may well incorporate security measures that limit unwanted interception of data, there is always the possibility that some third party may find a way of breaching the security measures.

One solution is to use an additional physical switch on the reader that activates and deactivates wireless functionality. A drawback of this option is that this increases the complexity of manufacture as well as cost of the device, and requires additional components in an already limited space.

It is desirable to improve the functionality of a test device, with the heavy cost constraints in mind.

### SUMMARY

An invention is defined in the claims.

According to an aspect, a system for changing a state of a reading device is provided. The system comprises: a reading device arranged to operate in a first state for reading a result of a test performed using a test stick, the reading device comprising a detection means arranged to detect a first reading influenced by a detection region of the test stick; an inserter for changing a state of the reading device; wherein, when the inserter is inserted into the reading device, the detection means detects a second reading distinct from the first reading, the reading device being arranged to operate in a second state in response to detecting the second reading.

Optionally, the inserter comprises an inserter region and, when the inserter is inserted into the reading device, the inserter region aligns with the detection means.

Optionally, the reading device further comprises a receiving region arranged to receive the test stick therein.

Optionally, the receiving region is also arranged to receive the inserter in place of the test stick.

Optionally, the inserter region of the inserter corresponds wholly or in part to the detection region of the test stick.

Optionally, the detection means comprises at least one light source for illuminating the detection region of the test stick and at least one photodetector for detecting light emanating from the detection region.

Optionally, the light emanating from the detection region is within a predefined light emanation range.

Optionally, the inserter region comprises at least one optical feature, and wherein, when the at least one light source illuminates the inserter region, the optical feature is such that light emanating from the inserter region falls outside the predefined light emanation range.

Optionally, the optical feature is a recess or hole.

Optionally, the optical feature provides the inserter region with a reflectance or transmittance different from that provided by the detection region.

Optionally, the reading device further comprises a switch, the switch being arranged to cause automatic activation of the at least one light source when the test stick or inserter is inserted into the reading device.

Optionally, the light emanating from the inserter region is reflected light or transmitted light or fluorescent light.

Optionally, when the test stick or the inserter is inserted into the reading device, the detection region or inserter region respectively is aligned with the at least one light source.

Optionally, the predefined light emanation range is a predefined light intensity range.

Optionally, the detection region comprises a test zone, the test zone being an area in which an assay test line develops.

Optionally, the inserter region corresponds to the test zone of the detection region.

Optionally, the detection region further comprises a control zone.

Optionally, the detection region further comprises a reference zone.

Optionally, the reading device is an assay result reading device.

Optionally, the reading device is an ovulation test device and/or a pregnancy test device.

Optionally, the reading device further comprises a wireless communication means, and wherein the wireless communication means is activated when the reading device operates in the second state.

Optionally, the wireless communication means is Bluetooth® or Bluetooth® Low Energy.

Optionally, the inserter is not a test stick.

Optionally, the inserter is a used test stick, and wherein an error notification is activated when the reading device operates in the second state.

Optionally, the error notification is provided by a display of the reading device.

Optionally, the error notification is provided by a light source on the reading device.

Optionally, the error notification is provided by a sound source of the reading device.

Optionally, the error notification indicates that the inserter is a used test stick.

Optionally, the reading device is arranged to read a result of a first test when in the first state and a result of a second, different test when in the second state.

Optionally, the test stick is a first test stick and the inserter is a second test stick.

Optionally, the first test stick is suitable for measuring at least one analyte and the second test stick is suitable for measuring at least one different analyte.

Optionally, the first test stick is suitable for measuring at least one analyte at a first sensitivity, and the second test stick is suitable for measuring the at least one analyte at a second sensitivity.

Optionally, the second sensitivity is greater than the first sensitivity.

Optionally, the system further comprises a second inserter for changing a function of the reading device, the second inserter comprising a second inserter region, wherein, when the second inserter is inserted into the reading device, the second inserter region aligns with the detection means such that the detection means detects a third reading distinct from the first reading and the second reading, the reading device being arranged to revert to operating in the first state in response to detecting the third reading.

Optionally, the second inserter is not a test stick.

Optionally, the first test is one of an ovulation or pregnancy test, and the second test is the other of an ovulation or pregnancy test.

According to another aspect, a method for changing a state of a reading device is provided. The reading device is arranged to operate in a first state for reading a result of a test performed using a test stick. The reading device comprises a detection means arranged to detect a first reading influenced by a detection region of the test stick. The method comprises the steps of: inserting an inserter into the reading device; and detecting, by the detection means, a second reading distinct from the first reading; wherein the reading device operates in a second state in response to detecting the second reading.

### BRIEF DESCRIPTION OF FIGURES

Embodiments of the invention will now be described, by way of example, with reference to the following drawings, of which:

FIG. 1 shows a perspective view of a known assay result reading device;

FIG. 2 shows example components located within the housing of the device of FIG. 1;

FIG. 3 shows an example arrangement of LEDs and photodetectors of the device of FIG. 1;

FIG. 4 shows a reading device in accordance with an embodiment;

FIG. 5 shows two inserters: a test stick and an activator in accordance with an embodiment;

FIG. 6 shows a flow diagram relating to a method of activating a wireless communication means of the device of FIG. 4;

FIG. 7 shows a flow diagram relating to a method of transmitting test result data from the device of FIG. 4 to an external device.

Throughout the description and drawings, like reference numerals refer to like parts.

### DETAILED DESCRIPTION

Disclosed herein is a system and method for changing a state of a device, such as an assay result reading device. The term “assay result reading device” means any device that detects biochemical content of a sample and outputs a result. In particular, an assay result reading device may be a digital ovulation test device for in-home use by a woman.

Such a test device may be used to determine the relative fertility of a woman at a certain point in her menstrual cycle. Other examples include digital pregnancy or menopause test devices.

In order to aid understanding of the invention, an example system will first be described. Such a system uses an assay result reading device and a separate test stick. The determination of fertility based on the assay result reading device and the test stick is achieved optically, as described in EP1484601B1. As an example, the assay result reading device is arranged to receive the test stick and thereafter provide an indication of a woman's fertility based on an optical analysis of a detection region of the test stick. The detection region of the test stick may also be termed a test strip. The assay result reading device comprises a light source arranged to illuminate a test zone of the detection region when the test stick is inserted and retained within the device, and a photodetector to detect the reflection of light from the detection region, or the transmission of light through the detection region.

The test zone is an area of the detection region which includes an area in which an assay line may develop. The assay result reading device may have more than one light source, for example first, second and third light sources. The detection region of the test stick may comprise additional zones. For example, additional test zones may be present in which the same or another analyte is determined. In some instances at least one additional zone known as a control zone may be present within the detection region of the test stick. The relative position of the test and control zones can be varied, with the control zone present either upstream or downstream of any test zone. In this example, having three light sources, each light source is arranged to illuminate a corresponding first, second and third zone. Each zone is a portion of the total area provided by the detection region of the test stick.

Each test zone may serve a different purpose, measuring the same or different analyte in the sample. In addition, the detection region may include areas or zones where there is no analyte measurement taking place, and these areas or zones may be interrogated by a measurement means to provide a reference zone for the detection region. The reference produced may be used to compensate for variations in the background colouration of the detection region which may vary between test sticks run with samples having varying colours, for example urine samples which can be concentrated due to dehydration for example where the sample is darker. Variations in running of the test stick can produce different degrees and variations in the rate of release of dried reagents, typically direct particulate labels such as dyed latex or colloidal gold sol, thereby producing variations in colouration of the background of the detection region. The reference zone can be used to compensate and account for such variations. For example, the first zone of the detection region may be a test zone, the second zone may be a reference zone, and the third zone may be a control zone. The test and control zones may be of any shape and size, and typically these are perpendicular lines relative to the length of the detection region/test strip.

The test zone is the zone in which accumulation or deposition of a label takes place, such as a particulate coloured binding agent, in response to the presence or absence of a particular analyte. For example, one analyte may cause a coloured line to appear in the test zone, such that a portion of the light reflected off or passing through this zone is absorbed. Other test devices may use alternative label and appropriate measurement means, for example electrochemical determination or use of fluorescent labels generating a fluorescent signal.

The control zone is the zone that acts as an experimental control. In this zone, a signal is formed irrespective of the presence or absence of the analyte of interest. This is to show that the procedure has been correctly performed and/or that the binding reagents are functional.

Calibration of the reading device may be performed in various ways, including calibration at the point of manufacture. Further calibration may take place during use of the test device to characterise the particular test stick being used. Calibration measurements may take readings from all or some of the zones within the detection region. All or some of the zones in the detection region may be used to validate the flow along the test strip. The reference zone may be used as a means to compensate for background signal resident on the test strip when it has been wetted with sample. An example calibration method is described in EP1484601B1, paragraphs [0041]-[0043].

In the case that only a single light source and only a single photodetector are used, the detection region may not be divided into different zones, and the entire detection region may serve the same function as the test zone. Alternatively, the test zone may be a defined region within the detection region. In the case that three light sources are used and the detection region is divided into three zones, the assay result reading device may comprise first and second photodetectors. The first photodetector is associated with the first light source, and may be located adjacent thereto. The first photodetector is arranged to detect light emanating from the first zone of the detection region. However, the first photodetector is so positioned as to detect light emanating from the second zone.

The second photodetector is associated with the third light source, and may be located adjacent thereto. The second photodetector is arranged to detect light emanating from the third zone of the test strip. However, the second photodetector is so positioned as to detect some of the light emanating from the second zone. In the case that the assay result reading device comprises a plurality of light sources, optical baffles may be provided between the light sources so as to help constrain the light from each light source to its respective zone, in combination with a microprocessor used to control which light sources are active in relation to specific photodetectors. Such an arrangement allows for the determination of results from three zones within the detection region by using two photodetectors, and presents a cost saving both in terms of component parts as well as simplifying manufacturing complexity.

It is also possible to use a plurality of light sources, each illuminating separate zones within the detection region, in conjunction with a single photodiode to detect light from each zone. In this case, the microprocessor controls the activation of the light sources as well as the detection by the photodetectors. Again, optical baffles are used to help constrain the light from each light source to its respective zone.

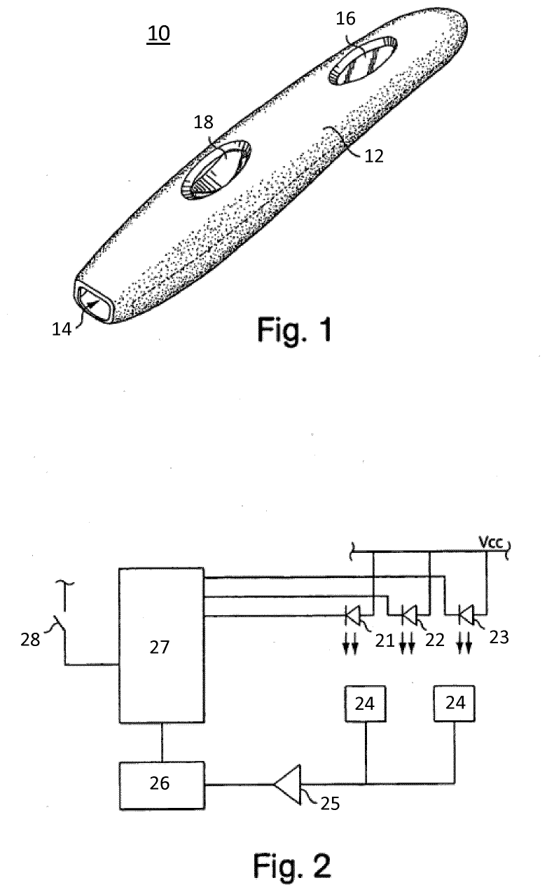

FIGS. 1-3 show an example assay result reading device and a test stick useful for understanding the invention. An example assay result reading device 10 is illustrated in FIG. 1. The reading device may be about 12 cm long and about 2 cm wide and is generally finger-shaped, however other dimensions and shapes may of course be used. The device 10 comprises a housing 12 formed from an opaque plastics material. The device has an aperture or insertion opening 14 at one end into which a test stick can be inserted. One face of the device 10 comprises an opening through which a display 16 may be seen. The display 16 can be any kind of conventional display, such as a liquid crystal display. The display 16 is arranged to provide information to a user of the device 10. The device also comprises ejection means 18 for ejecting a test stick from the device 10. The ejection means may be any suitable means, such as a push button arranged to eject a test stick from the device 10. The device 10 may also have an internal stopping abutment to limit insertion of the test stick into the device 10.

The test stick for use with the reading device is a generally conventional lateral flow test stick, for example of the sort disclosed in U.S. Pat. Nos. 6,156,271, 5,504,013, EP 728309, or EP 782707. In particular, the test stick having a strip of porous solid phase material as disclosed from page 6 line 24 to page 8 line 8 of EP291194B1 may be used. The test stick is sized and shaped to be insertable into the device 10, through the opening 14. The test stick is conventionally an elongated strip shape, however other shapes may be used.

FIG. 2 shows example components located within the housing 12 of the device 10. As mentioned, the device 10 may only have a single LED and a single photodetector, however the device shown in FIG. 2 has three LEDs and two photodetectors. The device 10 of FIG. 2 comprises a first LED 21, a second LED 22 and a third LED 23. When a test stick is fully inserted into the device 10 so as to abut the switch, each LED is aligned with the respective one or more zones of the detection region of the test stick. Two photodiodes 24 operate in the conventional manner: light is detected after reflection or transmission from each zone to generate a current, the magnitude of the current being proportional to the amount of light incident upon the photodiodes 24. In this example, the current generated is converted to a digital value by the microcontroller. Various other ways of converting the incident light exposed to the photodiodes are known in the art. In order to illuminate only one of the zones (primarily) at a given time, the microcontroller 27 switches the LEDs on individually, one at a time. The signals generated by reflected or transmitted light can therefore be attributed to a specific zone with the knowledge of when and which LED was switched on.

FIG. 2 also shows a switch 28. This switch 28 is an internal mechanical switch 28 located within the housing 12 of the device 10. Insertion of the test stick into the device 10 causes an abutment and activation of the switch 28. The activation of this switch “wakes” the device 10 from a “dormant” state into an active state by activating the microcontroller 27. The switch may additionally be positioned to perform the function of the internal stopping abutment to restrict the lateral movement of the test stick within the housing 12, meaning a separate stopping abutment is not provided. The device 10 also includes a power source for providing power to these components. Such a power source may be a battery, such as a coin cell battery for example.

An example method of using the assay result reading device 10 and a test stick to conduct an assay will now be described. At one end of the test stick is a sample receiving portion for receiving a sample to be analysed by the device 10. The sample receiving portion is typically located at an opposite end of the test stick to the end that would be inserted into the device 10. The sample receiving portion of the test stick is exposed to a liquid sample, typically urine, either before or after insertion of the test stick into the device. The exposure may be by placing the end of the test stick having the sample receiving portion into a urine sample pre-collected in a container or a urine stream from an individual for a duration of time, such as 5 seconds.

The device 10 then detects the intensity of light emanating from the detection region of the test stick. In other words, the device 10 detects the intensity of light reflected by or transmitted through the detection region of the test stick. Although reflected light is primarily referred to below, it is to be understood that the one or more LEDs and the one or more corresponding photodetectors may be located on opposite sides of the device 10. In this case, transmitted light is detected and the detection region must be transparent or translucent to allow light to pass from an LED, through the detection region and onto a photodetector.

In the case of the detection of reflected light, reflected light intensity from one or more of the zones of the detection region is then measured using the one or more photodetectors. The detection process may take place at a predetermined time interval following insertion of the test strip into the device 10, or may begin immediately. Measurements of light intensity may be taken multiple times, and averaging may be used to improve accuracy. Multiple measurements of light intensity may be taken over a period of time to provide a kinetic change of the light emanating from any of the zones to profile how the signal changes from any of the zones as a function of time. The LEDs used within the reader can be selected to emit a particular wavelength of light which is largely absorbed by the label of choice as it collects at the zones present in the detection region in an analyte dependent manner.

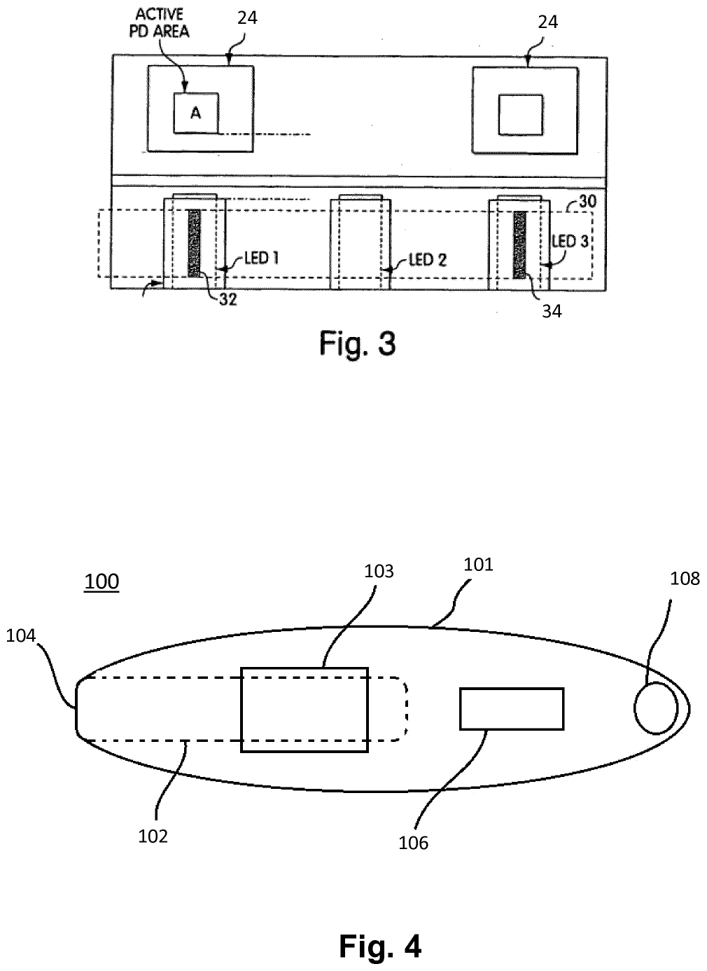

FIG. 3 shows an example arrangement of how three LEDs may be arranged with two photodetectors. Each photodetector has an active area (A) that is sensitive to light. The optical setup is arranged such that the centre lines of LEDs 1 and 3 correspond to the centre lines of the photodetectors 24. The LEDs and photodetectors shown in FIG. 3 may be located within an area of about 1 square cm. FIG. 3 also shows a detection region 30 of a test stick located above the three LEDs. The detection region 30 shown has a detection zone 32 and a control zone 34 which, when the test stick is inserted into the device 10, are located above LED 1 and LED 3 respectively.

The detection region 30 may be a known test strip comprising a layer of a porous carrier, such as nitrocellulose membrane, which may be adhered or cast onto a layer of plastic, such as MYLAR <®>. An additional plastic cover may be placed or adhered to the surface of the nitrocellulose membrane in totality or in part. The plastic layer of the detection region 30 that is proximal to the one or more LEDs must be transparent or translucent to allow light through. In the case that the one or more LEDs and the one or more photodetectors are located on the same side of the device 10, and therefore on the same side of the detection region 30 when the test stick is in the device 10, the plastic layer distal to the one or more LEDs must be capable of reflecting light. Preferably, the distal plastic layer is white to increase contrast and hence the signal to noise ratio. In the case that the one or more LEDs are located on an opposite side of the device 10 to the one or more photodetectors, i.e. the LED and photodetector are located on either side of the detection region 30, the plastic layers must both be transparent or translucent such that light can pass through the detection region 30.

It can be seen from the above description of FIGS. 1-3 that a known assay result reading device 10 is able to perform a test by receiving a test stick having a detection region 30, analysing the detection region 30 optically, and outputting a result on the display 16 of the device 10. The assay result reading device analyses the assay test lines that appear in the detection region and interprets the intensity of these assay test lines based on detecting the attenuation or transmission of light from the LEDs. One or more values indicative of the attenuation or transmission may be stored in a memory of the device. Measurement values from the test just completed are applied to an algorithm to determine a state of fertility. When performing additional tests, the results from the test just completed may be used in combination with all or some of the previous tests in an algorithm to determine the current state of fertility. Once a state of fertility is determined, a visual indication of the state of fertility is displayed on the display 16.

For example, a “peak” fertility state, representing a maximum fertility, may be displayed as a symbol such as a smiley face. Conversely, a “low” fertility state, representing a minimum fertility, may be displayed as a different symbol, such as a sad face or an empty circle. In this manner, the user is provided with an easily interpreted indication of their current fertility state. Additional or different fertility states and additional or different visual indicators of fertility may of course be used.

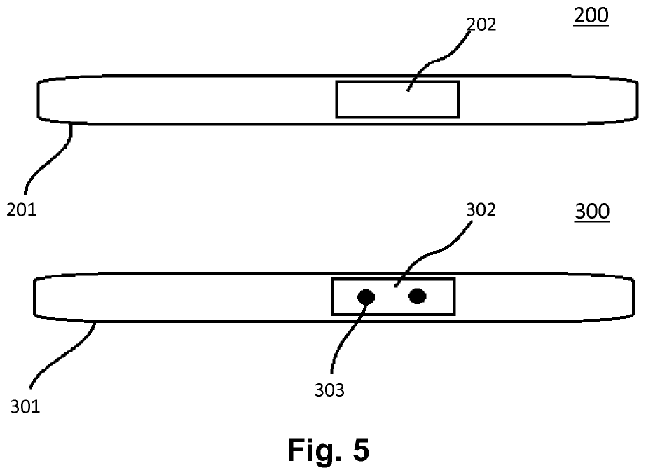

An embodiment will now be described in relation to FIGS. 4 and 5. FIG. 4 shows a reading device 100 and FIG. 5 shows two inserters: a test stick 200 and an embodiment of an activator 300. The reading device 100 may be the assay result reading device as described in relation to FIGS. 1-3. In FIG. 4, the device 100 has a body 101 and a receiving region 102 which is a cavity sized and shaped to receive the test stick 200 in its entirety or a portion of the test stick 200. The device 100 additionally has an insertion hole 104 creating an opening into the device 100 that allows the test stick 200 to enter into the receiving region 102. The device further comprises a display 106 for providing a visual indication of the state of fertility (as previously described), and optionally wireless communication means 108. The device 100 may also optionally have an ejection means (not shown). The ejection means may be any suitable means, such as a push button or lever arranged to eject the test stick 200 from the device 100. The device 100 may also have an internal stopping abutment (not shown) to limit insertion of the test stick 200 into the device 100.

The device 100 may additionally have a switch located at least partially within the receiving region 102. Insertion of the test stick 200 into the receiving region 102 causes an abutment of the test stick 200 and the switch, thereby activating the switch. Activation of the switch causes activation of a microcontroller as described above in relation to FIG. 2. Indeed, the device 100 includes the circuitry of FIG. 2 and is therefore activated in the same manner. The switch may additionally be positioned to perform the function of the internal stopping abutment, meaning a separate stopping abutment may not be provided.

The device 100 additionally comprises detection means 103 having at least one light source and at least one photodetector. The detection means 103 optically detects assay test lines, as previously described in relation to FIGS. 1-3.

The test stick 200 may be a conventional test stick having a lateral flow test strip (detection region). The test stick 200 comprises a body 201 and a detection region 202. The detection region 202 is the region of the test stick 200 having the one or more zones previously described, which may for example be the previously described test zone, the reference zone, and the control zone. The detection region 202 is located such that, when the test stick 200 is received in the receiving region 102, the detection region 202 aligns with the detection means 103.

Although three zones are discussed previously, any number of zones may be present, including only a single zone. The number and type of zones required depends on the specific application as well as the number of light sources and photodetectors used in the detection means 103. Indeed, there may only be one zone, the test zone, covering all or some of the detection region 202, and the detection means 103 may only have one photodetector and one light source.

The device 100 is arranged to operate in at least two states. In a first state, the device 100 is arranged to perform a first function, and in a second state the device 100 is arranged to perform a second, different function. The device 100 is able to automatically determine in which state to operate based on detecting emanating light from an inserter, as will be described. The word “inserter” is used to mean any object that may be inserted into the device 100. For example, the inserter may be a test stick in a different state, such as a used test stick, a different kind of test stick, or an activator that is not a test stick. The inserter may also be regarded as an article or object that can be inserted into the device 100.

In an embodiment, the device 100 as able to distinguish between an unused test stick 200, and a used test stick 200. A used test stick is one to which a sample has already been applied, and assay lines have already developed completely or partially in the detection region 202. Light emanating from the detection region 202 of an unused test stick 200, when the test stick 200 is in the receiving region 102 and the detection means 103 is operating, emanates within a predefined light emanation range. The value of the predefined light emanation range is set by software of the device 100. The predefined light emanation range may be any detectable characteristic of the emanating light. For example, intensity, frequency or another light characteristic may be used. The predefined light emanation range may be a predefined light intensity range.

In this embodiment, light emanating from an unused test stick 200 may have a predefined light intensity range. In the case of the emanating light being reflected light, the predefined light intensity range may be, for example, between 75-125% of the light reflected by a calibration stick during manufacture (the calibration value). The light intensity value detected by the detection means 103, in the case of the emanating light being reflected light from an unused test stick, may therefore be between 75-125% of the calibration value. The range of between 75-125% light intensity is purely an example, and different ranges would be used depending on the particular device and the particular test stick. For an embodiment in which the detection region 202 has more than one zone, predefined light intensity ranges may be specified corresponding to each zone or to some of the zones. In the embodiment having a detection region with three zones, the predefined light intensity ranges for an unused test stick 200 may be as follows. A first zone may have a predefined light intensity range of between 75-125%, a second zone may also have a predefined light intensity range of between 75-125%, and a third zone may have a predefined light intensity range of between 75-125%.

Alternatively, the predefined light intensity range may be a percentage light intensity range of reflected light compared to that output by the detection means 103. For example, between 50-75% of the light output by the detection means 103 may be reflected back at the detection means 103. Again, other ranges may of course be used depending on the specifics of the inserter.

When an unused test stick 200 is inserted into the receiving region 102, software on the device 100, via a microprocessor, determines that the light emanating from the detection region 202 falls within the predefined light intensity range or ranges corresponding to light emanating from an unused test stick. The device 100 therefore determines that an unused test stick 200 is inserted. In the case of multiple zones as described above, the software may require that the predefined light intensity ranges of all zones are satisfied before making the determination that an unused test stick 200 has been inserted. This is to account for the fact that some of the predefined light intensity ranges may be satisfied during the normal running of a test or in scenarios in which an unused test stick 200 is not inserted. However, the requirement to satisfy multiple predefined light intensity ranges ensures greater accuracy in determining that an unused test stick 200 has been inserted.

Accordingly, the device 100 is able to determine that an unused test stick 200 specifically is in the receiving region 102 when light within the predefined light emanation range(s) corresponding to an unused test stick 200 is detected by the detection means 103. In response to making this determination, the device 100 operates in a first state in which analysis of the results of a sample applied to the unused test stick 200 is performed, as previously described. In the event that the device 100 is not already in the first state, the device 100 automatically changes state to the first state in response to making this determination.

The device 100 is therefore able, using light, to analyse a test stick to provide a test result as previously described, and the device 100 is also able to determine that an unused test stick 200 has been inserted into the device 100.

In an embodiment, the device 100 is able to distinguish between unused and used test sticks. In some instances, users are known to use an unused test stick in the reader on one occasion and then, knowingly or not, try and re-use the same test stick (i.e. now a used test stick) in the device on a subsequent occasion. This can cause errors in the testing regime, in particular when tests are run on successive days where results are used to build up a profile of the analyte(s) over a period of time. Known software safeguards can be used to prevent a used test stick from being reused, and therefore read, by a test device. These safeguards operate by monitoring and validating the flow (as the test strip/detection region wets with sample) as well as monitoring the development of assay strip signal, however these known safeguards typically require the device to read the test for the full operating time of the test before confirming the test is invalid. By reading the test for the full operating time, the device unnecessarily uses battery power to generate an output that is invalid.

To solve this problem, in this embodiment the device 100 beneficially automatically changes state to quickly alert the user that the test stick 200 inserted has already been used, before running the test for an unnecessarily long time. As described above, the device 100 is able to quickly determine, via a microprocessor, that an unused test stick 200 has been inserted based on the predefined light intensity range or ranges being satisfied. In the event that a used test stick 200 is inserted however, the light emanating from the detection region 202 of the used test stick 200 would not satisfy the predefined light intensity range(s). This could be for a number of different reasons.

As one example, on insertion of a test stick 200, the measurement of emanating light intensity as a result of a signal (assay line) from the control zone of the detection region 202 can be compared to a predefined light intensity range corresponding to a signal stored at manufacture. If the emanating light intensity is below a certain intensity threshold of the predefined light intensity range, this indicates a control line is already present in the control zone of the detection region 202 (i.e. the test stick has already been used) and the device 100 is therefore able to determine that the test stick 100 is a used test stick.

As another example, certain assay formats used to measure an analyte can be optimised such that they generate a certain signal (assay line) at the test zone of the detection region 202 at all analyte levels tested. An example of this is competition or hapten assays, which are commonplace on Lateral Flow Assays. In this example, the absence of an analyte produces a strong assay line at the test zone since antibody coated labels, such as gold sol, bind to an analyte or analyte-analogue conjugate immobilised at the test zone. As the level of analyte in the sample increases, it occupies sites on the antibody coated label and prevents the label binding at the test zone, thereby reducing the signal seen at the test zone. Increasing analyte levels tested (i.e. applying a sample to the test stick) hence reduces the signal at the test zone. The assay can so be optimised such that there are sufficient antibody sites on the label which are unbound by analyte at the highest level of analyte possible in a physiological sample. As such, an assay line, albeit reduced in intensity, is produced at the test zone at the highest possible analyte concentration anticipated to be present in a sample. The reduced signal at the test zone results in a light intensity change that can be detected by the device 100 and used in a similar manner as the control zone described above to differentiate a used test stick from an unused test stick.

In short, when a used test stick 200 is inserted, the light emanating from the detection region 202 of the used test stick 200 does not satisfy the predefined light intensity range(s) corresponding to an unused test stick. This may be by exceeding or falling below a certain light intensity threshold, or by falling outside of the range. Alternatively, the device 100 may also have a predefined light intensity range or ranges corresponding to a used test stick 200. As a result, software on the device 100 determines that the range(s) corresponding to an unused test stick 200 is not satisfied, or the range corresponding to a used test stick 200 is satisfied, and therefore an unused test stick 200 is not inserted (or a used test stick 200 is inserted). In response to determining that an unused test stick 200 is not inserted or a used test stick is inserted, the microprocessor of the device 100 stops the testing process and automatically changes the device 100 from the first state to a second state. The second state is the state appropriate for when a used test stick 200 has been inserted into the device 100. In the second state, the device 100 generates a notification to the user. The notification may inform the user that the test stick 200 has already been used. Such a notification may take the form of text or a symbol on the display 106, or the device 100 may emit a sound or emit light. For example, a light source, such as an LED, may illuminate, or a sound source, such as a speaker, may emit a sound. These are merely examples of how the user may be made aware of the notification, however other methods may be used.

In another embodiment, in a first state the device may operate as an ovulation testing device, and in a second state the device may operate as a pregnancy testing device. For such a multi-purpose device, a manner of changing from the first state to the second state is provided as will be described.

In this embodiment, the device 100 may be able to determine whether a first test stick designed to measure analytes for determining a fertility status has been inserted, or whether a second test stick designed to measure analytes for determining a pregnancy status has been inserted. The first test stick may therefore measure LH and E3G to provide an indication of the status of ovulation. The second test stick may measure human chorionic gonadotropin (hCG) to provide an indication of the status of pregnancy. On determining that the second test stick has been inserted, the device 100 may change from the first state to the second state, so as to be suitable for reading the results of a pregnancy test.

In this embodiment, the first test stick and the second test stick each have a detection region 202 as described above for the unused test stick 200, and each detection region 202 is also read by the device 100 in the same manner as described above for the unused test stick 200. However, the detection region 202 of the second test stick has at least one second optical feature, and the detection region 202 of the first test stick may optionally also have at least one first optical feature.

In the case that the detection region 202 of either stick is transparent or translucent, the optical feature (of one or both test sticks, whichever has it) is such that light passing through the detection region 202 is affected or influenced in a way that is different for the first test stick compared to the second test stick. In the case that the detection region 202 is not transparent or translucent, the optical feature is such that light incident on the detection region 202 is reflected in a way that is again different for the first test stick compared to the second test stick. Therefore, the optical feature serves to ensure that light emanating from the first test stick is different to light emanating from the second test stick. It may be the case that the detection region 202 of the first test stick is transparent or translucent, and the detection region 202 of the second test stick is not transparent or translucent, or vice versa.

The device 100 may have a first predefined light emanation range corresponding to the first test stick. Therefore, when the first test stick is inserted in the device 100, the device 100 is able to determine that the first test stick has been inserted as light emanating from the first test stick falls within the first predefined light emanation range. In response to this determination, the device 100, via a microprocessor, operates in or changes to the first state to perform ovulation testing. The first state is therefore the state appropriate for reading the results of an ovulation test, which may be by applying a specific algorithm appropriate for ovulation testing.

In the case that the second test stick is inserted, the device 100 is able to determine that the first test stick is not inserted as light emanating from the second test stick falls outside the first predefined light emanation range. Alternatively, the device 100 may have a second predefined light emanation range corresponding to the second test stick, and the determination is based on the light emanating from the second test stick falling within the second predefined light emanation range. In response to this determination, the device 100, via a microprocessor, operates in or changes to the second state to perform pregnancy testing. The second state is therefore the state appropriate for reading the results of a pregnancy test, which may be by applying a specific algorithm appropriate for pregnancy testing.

Depending on the specifics of the optical feature of one or both test sticks and whether the light is reflected or transmitted, the light emanating from the detection region 202 of the second test stick, for example, may have a light intensity less than the lower limit of the first predefined light emanation range, or greater than the upper limit of the first predefined light emanation range. Conversely, the light emanating from the detection region 202 of the first test stick, for example, may have a light intensity less than the lower limit of the second predefined light emanation range, or greater than the upper limit of the second predefined light emanation range.

Example optical features will now be described. The optical feature 303 may be a hole in the detection region 202. In the case that the LED and photodiode of the detection means 103 are located on opposite sides of the receiving region 102 (i.e. light must pass through an object inserted in the receiving region 102 to be detected), more light passes through the detection region 202 than would be possible without that particular optical feature 303. More specifically, more light is transmitted through the detection region 202 having a hole than a detection region 202 without a hole. In the case that the second test stick has a hole optical feature and the first test stick doesn't, the light detected by the detection means 103 therefore exceeds the upper limit of the first predefined light emanation range.

Following the same example, in the case that the optical feature is a hole and the LED and photodiode of the detection means 103 are located on the same side of the receiving region 102 (i.e. light must be reflected off an object inserted in the receiving region 102 to be detected), when the second test stick 200 is inserted in the receiving region 102, less light may be reflected from the detection region 202 of the second test stick 200 than would be possible for the detection region 202 of the first test stick 200 (which doesn't have a hole). The detected light emanating from the second test stick therefore does not meet the lower limit of the first predefined light emanation range.

Further examples of the different optical feature(s) of one or both test sticks are now described. As an example, the porous carrier of the detection region 202 of each test stick may have a dye or label, such as latex or gold sol, deposited onto the porous carrier in a position aligned in the region of one or more of the zones within the detection region 202. On insertion of a dry, (unused) test stick into the device 100, a specific attenuation of light is produced from these zone(s), informing the device 100 which type of stick has been inserted into the reader since tests for pregnancy may attenuate the light differently to those for ovulation. On running the test stick after a sample has been applied, the dye or label becomes mobile and gets washed away with the flow of sample. A signal (which may be one or more of the test, reference or control signal) may subsequently develop in this region as the test runs. In this way, a light emanation is detected by the device to differentiate the type of test stick, and the relevant part of the detection region 202 then becomes clear as the label is washed away. Further signals related to the assay may well develop as the test runs. Different amounts of dye or label may be deposited to distinguish the different types of test stick, (for example to differentiate pregnancy test sticks from ovulation test sticks). The different amounts of dye or label may be formed for example by varying the concentration of material deposited or the width of a line.

As another example, the dye or label described above does not wash off as the test is run. Instead, a line, for example, could be deposited on the nitrocellulose or the plastic Mylar backing in a position which is within the area of any zone(s) on the test strip (detection region 202). In this case, the line may be offset in the window. Different intensities/colours of line could be used to discriminate one type of test stick from another. On reading, the device 100 has an offset when the dry, (unused) test strip is placed in the reader, the offset defining which type of test stick is being used. The test signal may develop alongside this line, (or over this line) resulting in a change in signal from the zone(s) on the test strip (detection region 202).

As another example, the backing material (Mylar) on which the membrane (for example nitrocellulose) resides may be of different colours or thicknesses in the first test stick compared to the second test stick. Again, this creates two different offsets in light attenuation informing the device 100 as to which type of test stick is being used.

As another example, each test strip may have different sized holes in the region of one or more of the zones in the detection region 202. The different hole sizing causes attenuation of the light differently for the first test stick compared to the second test stick. The hole(s) may align to the centre of any zone and the test line could form at either side of this hole. The hole may for example extend through the membrane as well as the Mylar, or may only extend through the membrane and not the backing material by removing the membrane in a defined area away from the backing material, for example by scrapping or other means.

As another example, part of the moulding forming each test stick may extend differently into the vicinity of any of the zones of the detection region 202 for the first test stick compared to the second test stick, thereby influencing the attenuation of light differently between types of test stick.

In another embodiment, the changes of state from the first state to the second state may not be caused by insertion of the first and second test sticks. Instead, one or more inserters such as an activator 300 as shown in FIG. 5 may be used. A first activator 300 may be used for changing the device 100 to the first state, and a different, second activator 300 may be used for changing the device 100 to the second state. Each activator 300 is also configured to be inserted into the receiving region 102 in the same manner as the test stick 200. Insertion of the activator 300 also therefore causes activation of the switch in the same manner. The activator 300 has a body 301 and an activator region 302 corresponding at least in part to the detection region 202 of a test stick.

In this example, it is the activator region 302 of the first activator 300 that comprises the at least one first optical feature 303, and the activator region 302 of the second activator 300 that comprises the at least one second optical feature. The optical feature of each activator is positioned in a corresponding position to the detection region 202 of the test stick 200. More particularly, each activator region 302 may be positioned in a corresponding position to the test zone of the detection region 202. Each activator region 302 may be positioned in a corresponding position to the reference or the control zone or indeed in a position corresponding to more than one zone of the detection region 202. Although two optical features 303 are shown in the activator 300 of FIG. 5, any number of optical features may be present, including only a single optical feature 303.

The optical features 303 of each activator 300 have the same effect as those previously described for the first and second test sticks. For example, when the first activator 300 is inserted, the device 100 is able to determine that the first activator 300 has been inserted as light emanating from the first activator 300 falls within the first predefined light emanation range. In response to this determination, the device 100, via a microprocessor, operates in or changes to the first state to perform ovulation testing. Similarly, in the case that the second activator 300 is inserted, the device 100 is able to determine that the first activator 300 is not inserted as light emanating from the second activator 300 falls outside the first predefined light emanation range. Alternatively, the device 100 may have a second predefined light emanation range corresponding to the second activator 300, and the determination is based on the light emanating from the second activator 300 falling within the second predefined light emanation range. In response to this determination, the device 100, via a microprocessor, operates in or changes to the second state to perform pregnancy testing.

The first and second activators 300 are therefore used to change the state or mode of the device 100. The appropriate first or second test stick is subsequently inserted into the device 100 after the state of the device 100 has been changed by the corresponding activator 300. For example, if the user wishes to test for pregnancy via the second test stick, the second activator 300 is inserted into the device 100 to change the device 100 into the second state, and then the second test stick for pregnancy testing is inserted. To change back to the first state, the first activator 300 is then inserted into the device 100 and the first test stick may subsequently be inserted for ovulation testing.

In this embodiment, the device 100 is therefore able to automatically change from a first state in which a first condition is tested, and a second state in which a second, different condition is tested. This may be automatically achieved via the test sticks themselves, or via specific activators. Although the examples of fertility and pregnancy are used, any two different conditions may be used along with appropriate test sticks. Other analytes indicative of the health or wellbeing of the user may be tested in a similar fashion where a first test stick is used to ascertain a first measurement or reading of an analyte(s) and a second test stick is used to determine a measurement or reading of a second analyte(s), the first and second analyte(s) being different and being used to assess different heath conditions.

The first and second test sticks may in fact measure the same analyte, however the sensitivity (detection limits) may be different. For example, in the case of pregnancy testing, the first test stick may be very sensitive to low levels of hCG and therefore be used in the early stages of pregnancy to establish the presence of pregnancy, (a high sensitivity test). The second test stick may be used to detect higher levels of hCG which are apparent in the later stages of pregnancy and are useful for studying the progression of pregnancy (a low sensitivity test). The first state may therefore test for a condition at a first, higher sensitivity, and the second state may test for the condition at a second, lower sensitivity. Using different test sticks to measure the same analyte at different sensitivities is very useful since it can be difficult to measure the same analyte at different concentrations on the same test stick.

In another embodiment, in a first state the device may operate to read the results of a test, and in a second state the device may activate a wireless communication means. In this embodiment, the device 100 comprises wireless communication means 108 that may be beneficially used to transmit such a test result to an external device. This functionality requires a manner of initially activating the wireless communication means 108, while taking into consideration the need to minimise complexity and cost of the device 100. To this end, an activator 300 is provided as a method of changing the state of the device to the second state and causing automatic activation of the wireless communication means 108 without having to make any further hardware changes to the device 100. For example, no additional switches or other components are required to be added to the device 100, as the activator is able to cause activation of the wireless communication means 108 in the manner described below.

The activator 300 is as described above for the first activator or the second activator. In the case that the activator region 302 is transparent or translucent, the optical feature 303 is such that light passing through the activator region 302 is affected or influenced in a way that would not be possible on insertion of the test stick 200, regardless of the state of the detection region 202 of the test stick 200 (e.g. wet, dry or displaying a test result). In the case that the activator region 302 is not transparent or translucent, the optical feature 303 is such that light incident on the activator region 302 is reflected or influenced in a way that again would not be possible were the test stick 200 inserted instead.

As described above for other embodiments, due to the one or more optical features 303 of the activator region 302, when the activator 300 is in the receiving region 102, light emanating from the activator region 302 falls outside the predefined emanation range corresponding to an unused test stick. The light emanating from the activator region 302 may also fall outside the predefined emanation range corresponding to a used test stick. The optical feature 303 may be as described above, for example a hole, reflective element or any other element influencing light emanation in a unique or different manner.

Therefore, as the light emanating from the activator 300 falls outside the predefined light emanation range, software on the device 100 determines that a test stick is not inserted in the receiving region 102. Alternatively, as for the other embodiments, the device 100 may have a predefined light emanation range corresponding to the activator 300, and software on the device 100 may determine that the activator 300 is inserted based on light emanating from the activator 300 falling within the predefined light emanation range corresponding to the activator 300. Based on this determination, the device 100 therefore determines that the activator 300 must be inserted in the receiving region 102 and the device 100 automatically changes state, via a microprocessor, to the second state in which the wireless communication means 108 is activated.

Removal of the activator 300 may result in the device 100 returning to the first state and the wireless communication means 108 immediately being deactivated. Such a deactivation may be caused by the software no longer detecting the presence of the activator 300. Alternatively, removal of the activator 300 may cause the wireless communication means 108 to deactivate after a certain amount or type of data has been transmitted, or for a set duration after removal, for example 5 seconds. Other time durations may be used. Deactivation of the wireless communication means 108 may be caused by the switch no longer being activated in the absence of the activator 300.

The wireless communication means 108 may be any means of wireless transmitting data, for example Bluetooth®. In particular, Bluetooth® Low Energy (BLE) may be used to minimise the impact on battery life of the device 100. As the skilled person would understand, other wireless communication means may be used. For example, Wi-Fi, NFC, ZigBee® and ANT are just examples of other known wireless communication means that may be used.

In any of the above embodiments, the predefined light emanation ranges may be predefined light intensity ranges. “Falling outside” the predefined light emanation range may mean exceeding an upper limit of the light emanation range, or failing to meet a lower limit of the light emanation range. Any of the predefined light emanation ranges may instead be predefined light emanation thresholds, and therefore “falling outside” may mean exceeding a predefined light emanation threshold, or failing to meet a predefined light emanation threshold.

As the skilled person would understand, the optical feature in any of the embodiments may have many different properties, and may not be a hole. The only requirement for the optical feature is that light emanating from the detection region 202 or activator region 302, as appropriate, is influenced in such a way as to fall outside a certain predefined light emanation range(s). In other words, the light incident on the detection region 202 or activator region 302, as appropriate, is affected or influenced in a manner that is not possible in the absence of the optical feature.

For example, the optical feature may be a material more reflective than any material of the detection region 202 of a test stick from which to be differentiated, or the test zone of the detection region 202 of such a test stick, in its dry or wet state after a sample has been applied, or conversely may be a material less reflective than any material of the detection region 202 of a test stick from which to be differentiated or the test zone of such a test stick, even when excessive quantities of label are present at the test zone(s) resulting in the test zone(s) assay lines being at their maximum intensity (i.e. most dark). For example, the optical feature may be a dark or black shape, such as a spot. Alternatively, the optical feature may be a physical feature such as a raised area which has a shape and/or surface imparting specific reflective or transmissive properties. The optical feature may be a filter having a specific optical property which influences light from the LEDs in a manner that would not be possible by the detection region 202 of a test stick 200 from which to be differentiated.

The shape and size of the body 301 of the activators 300 previously described may correspond to the shape and size of the body 201 of the test stick 200. This may not be the case however, and the only requirement is that, when all or part of the activator 300 is inserted into the receiving region 102, the activator region 302 aligns with the detection means 103 and the switch is activated.

By using any of the above methods to change the state of the device 100, there is no need to include a manual switch on the device 100 to change the state. This results in a simpler and cheaper device that does not require any additional components, and further the same device 100 may be used to read multiple types of test sticks or activators.

In the case of a device 100 having wireless communication means 108, the user is able to selectively control the wireless communication function at will by simply inserting the activator 300 into the device 100. This presents a considerable power efficiency improvement since the wireless communication means 108 is not active while the detection means 103 is analysing the detection region 202. As a result of the above, power consumption is saved, which is particularly important for devices with limited battery life, particularly those designed for reading a number of test sticks, typically over consecutive days.

Assay result reading devices of the type described herein are disposable, and designed for a limited number of uses. For example, such devices may be designed to have sufficient power for several months' use. As such, any manner of reducing the cost and complexity of these disposable items, and reducing the power consumption, is very beneficial in this area of technology.

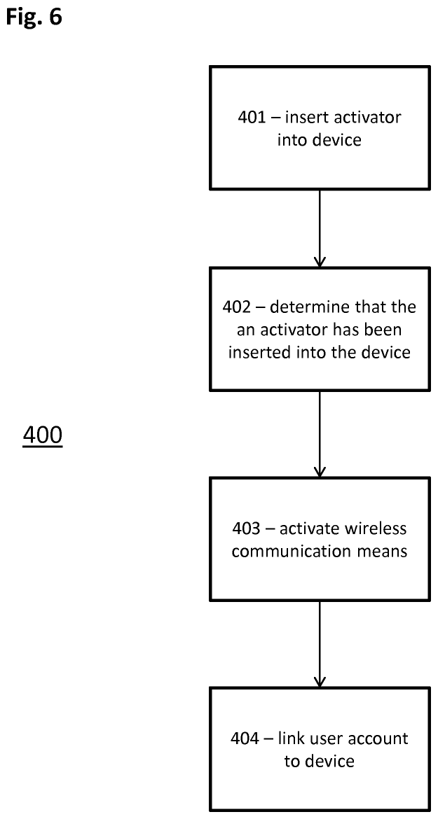

A method 400 of activating the wireless communication means 108 of the assay result reading device 100, and registering or “linking” the device 100 to a user account on an external device for the first time, will now be described with reference to FIG. 6.

At step 401, the user inserts the activator 300 into the device 100, via the insertion hole 104, such that the activator region 302 is aligned with the detection means 103 and the internal switch is activated.

At step 402, the detection means 103 determines from the measurements of emanating light (reflected or transmitted light) that an activator rather than a test stick is inserted. As previously described, the one or more optical features 303 of the activator region 302 result in light emanating that falls outside the predefined light emanation range and could not have been caused by the test stick.

At step 403, as it has been determined at step 402 that the activator 300 has been inserted, the wireless communication means 108 is activated. The wireless communication means 108 is only activated for a limited duration, for example less than 20 seconds, however other durations may be used, for example a number of minutes.

At step 404, an external device having a corresponding wireless communication means is connected with the device 100 to register or “link” the device 100 to a user account. Using software on the external device, such as an “app”, the user will have, prior to this step or at step 404, created a user account unique to the user. The user account may have been created using a conventional registration process involving a username and password. Once the user account has been created, the user account has a unique identifier that is unique to that account. The unique identifier may be anything that is able to distinguish the user in question from another user, for example the username or a generated sequence of letters, numbers, symbols or a combination of these. Registration or “linking” of the device 100 with the external device at step 404 involves the external device transmitting the unique identifier to the device 100. The unique identifier is then stored in a memory of the device 100 for later use, and the device 100 is regarded as registered or “linked” to the user account.

In any subsequent wireless communication to transmit results data from the device 100 to an external device, as a first step the device 100 requests a unique identifier from the external device. After receiving the unique identifier from the external device, the device 100 checks to see whether the received unique identifier matches the unique identifier stored in the memory of the device 100. If the two unique identifiers match, results data is transmitted from the device 100 to the external device. In this manner, the device 100 only transmits results data to an external device running the user's registered account. The results data transmitted may comprise the test result alone or in combination with other data related to the running characteristics of the device 100 such as flow time, control line results and other characteristics. Software updates may be sent from the external device to the device 100.

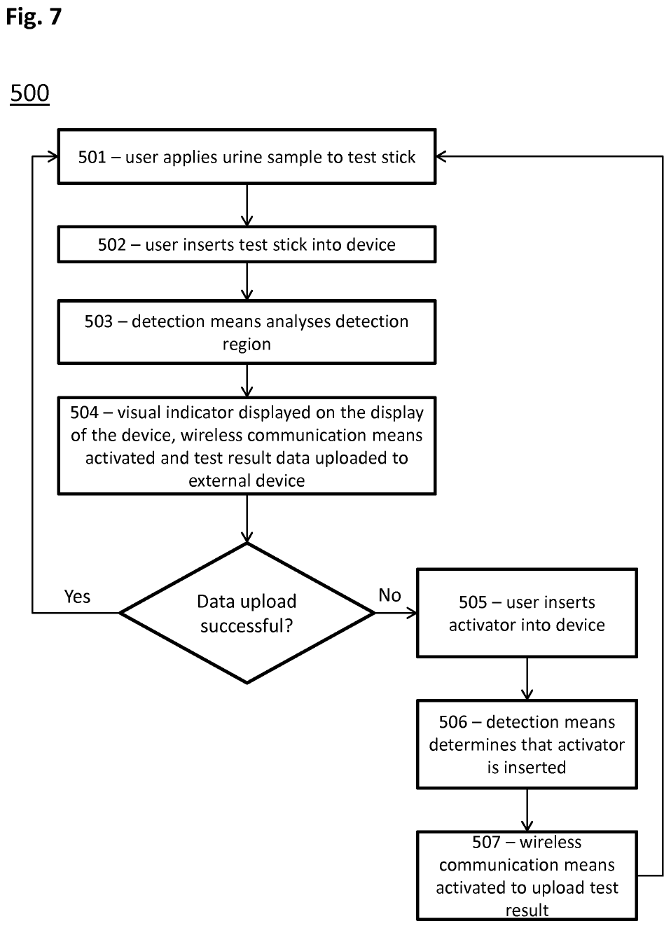

A method 500 of transmitting results data from the device 100, after the device 100 has been linked to a user account as described in the method 400, will now be described with reference to FIG. 7.

At step 501, a user applies a urine sample to the test stick 200. After the urine sample is applied, assay test lines begin to appear in the detection region 202 as previously described. Any or all of the intensity, colour or location of the assay test lines is indicative of a certain concentration of compound(s) in the urine sample, and therefore a certain fertility state of the user.

At step 502, the user inserts the test stick 200 into the device 100, via the insertion hole 104, such that the detection region 202 is aligned with the detection means 103 and the internal switch is activated.

Alternatively, step 502 may precede step 501. In other words, the user may insert the test stick 200 into the device 100 before applying the urine sample.

At step 503, the detection means 103 of the device analyses the detection region 202 to determine a test result. The analysis is performed using a combination of LED(s) and photodetector(s), as previously described. The detected intensity of light received at the photodetector(s) is measured, as previously described, and one or more values may be stored in a memory of the device 100. The number of LEDs and photodetectors may vary depending on the number of assay lines to be detected, and a single LED and a single photodetector may be used for simpler systems. An algorithm is then applied to the measurement to determine a state of fertility. The measurement may be applied to an algorithm in combination with stored values derived from previous test measurements to determine a state of fertility, as previously described. The state of fertility forms at least part of the test result data. The test stick 200 may then be removed and discarded.

At step 504, a visual indicator is displayed on the display 106 to provide an indication to the user of their current state of fertility based on the output of the algorithm. Further, if the device 100 is already linked with a user account as described in relation to the method 400, then at step 504 the device 100 also activates the wireless communication means 108 to upload or transmit the test result data relating to the output of the algorithm to an external device. As previously described, the test result data is only uploaded to the external device if the external device is operating the user account linked to the device 100. The upload of the test result data from the device 100 to the external device may be performed in any conventional manner. The wireless communication means 108 is only activated at step 504 for a limited duration, for example less than 20 seconds, to minimise the effect that the wireless communication means 108 has on the battery life of the device 100. Other durations may of course be used, with shorter durations having a further reduced effect on the battery life.

Optionally, at step 504, the visual indicator may not be displayed on the display 106.

If the test result data is successfully uploaded to the external device, the wireless communication is disabled and the method 500 then returns to the beginning of the method 500 at step 501 then step 502, or step 502 then step 501 as the order of steps 501 and 502 may be reversed.

If, at step 504, the upload of the test result data from the device 100 to the external device is unsuccessful, for example not completed within the time that communication is enabled, then the method proceeds to step 505. The upload of the test result data may be unsuccessful for various reasons. For example, corresponding wireless communication means of the external device may not be active during the period in which the wireless communication means 108 of the device 100 is active, or the external device may not have been close enough to the device 100. At step 505, the user inserts the activator 300 into the device 100, via the insertion hole 104, such that the activator region 302 is aligned with the detection means 103 and the internal switch is activated.

At step 506, the detection means 103 determines from the measurements of emanating light (reflected or transmitted light) that an activator rather than a test stick is inserted. As previously described, the one or more optical features 303 of the activator region 302 result in light emanating that could not have been caused by the test stick.

At step 507, as it has been determined at step 506 that the activator 300 has been inserted, the wireless communication means 108 is activated to upload the test result data to the external device. Again, the wireless communication means 108 may only be activated for a limited duration, for example less than 20 seconds, however other durations may be used.

Again, if the test result data is successfully uploaded to the external device, the wireless communication is disabled and the method 500 then returns to the beginning of the method 500 at step 501 then step 502, or step 502 then step 501 as the order of steps 501 and 502 may be reversed.

The external device is arranged to receive the transmission. Such an external device may be any wireless communications device with the capability of communicating with the wireless communication means 108. For example, a mobile phone or tablet with Bluetooth® capability may be used. Software running on the wireless communications device may be arranged to receive the data from the device 100 and prepare the data for visual representation. Such software may be an “app” resident on the wireless communications device. The software may provide an indication of fertility to a user by outputting a visual indication on a display of the external device. The communication between the device 100 and the external device may be encrypted in a conventional manner.

The activator 300 may be used with the assay result reading device 100 before insertion of any test stick 200, as part of a registration process as described above. The user may choose to activate the wireless communication means 108 and register the device 100 to their external device account whenever they wish. If a number of tests have been performed by the device 100 before registration, i.e. a number of test sticks have been run, the device 100 records the test results and any other relevant information until the time when the wireless communication means 108 is activated, whereupon the transfer of results data from the device 100 to the external device operating the user account occurs.

As can be seen, the ability of the device 100 to recognise different inserters, be it activators or test sticks, allows automatic state changing and therefore enhanced capabilities of the device 100. Such enhanced capabilities do not require any additional hardware as the capabilities are based on an automatic comparison of detected emanating light with predefined light emanation ranges or thresholds. A microprocessor of the device 100 can therefore automatically change the device 100 into a different state based on the result of the comparison.

The described embodiments are particularly beneficial in the case that it is desirable to minimise the production costs of the device 100. Such a device may be an assay result reading device such as an ovulation or pregnancy test, which is typically designed to be disposable and have a limited battery life. The described embodiments avoid the need for any new hardware features to be implemented into the device 100, and instead the existing functionality of the device (optical detection via one or more LEDs and one or more photodetectors) may be used to determine whether a state change should be made. For example, the existing functionality can be used to determine when an activator 300 has been inserted and to activate the wireless communication means 108 of the device accordingly.

Aspects of the various methods described above may be implemented by a computer program product. The software resident on the testing device and the external device is an example of such a computer program product. The computer program product may include computer code arranged to instruct the devices to perform the functions of one or more of the various methods described above. The computer program and/or the code for performing such methods may be provided to an apparatus, such as the devices, on a computer readable medium or computer program product. The computer readable medium may be transitory or non-transitory. The computer readable medium could be, for example, an electronic, magnetic, optical, electromagnetic, infrared, or semiconductor system, or a propagation medium for data transmission, for example for downloading the code over the Internet. Alternatively, the computer readable medium could take the form of a physical computer readable medium such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

An apparatus such the devices may be configured in accordance with such code to perform one or more processes in accordance with the various methods discussed herein. In one arrangement the apparatus comprises a processor in additional to a memory, and a display. Typically, these are connected to a central bus structure, the display being connected via a display adapter. The devices, particularly the external device, can also comprise one or more input devices (such as a mouse and/or keyboard) and/or a communications adapter for connecting the apparatus to other apparatus or networks. In one arrangement, the database resides in the memory of the devices. Such an apparatus may take the form of a data processing system. Such a data processing system may be a distributed system. For example, such a data processing system may be distributed across a network.

1-72. (canceled)

73. A system for changing a state of a reading device, the system comprising:
a reading device arranged to operate in a first state for reading a result of a test performed using a test stick, the reading device comprising a detection means arranged to detect a first reading influenced by a detection region of the test stick; and
an inserter for changing a state of the reading device;
the system being configured such that when the inserter is inserted into the reading device, the detection means detects a second reading distinct from the first reading, the reading device being arranged to operate in a second state in response to detecting the second reading.

74. The system of claim 73, wherein the inserter comprises an inserter region, configured such that when the inserter is inserted into the reading device, the inserter region aligns with the detection means.

75. The system of claim 73, wherein the reading device further comprises a receiving region arranged to receive the test stick therein.

76. The system of claim 75, wherein the receiving region is also arranged to receive the inserter in place of the test stick.

77. The system of claim 74, wherein the inserter region of the inserter corresponds wholly or in part to the detection region of the test stick.

78. The system of claim 72, wherein the detection means comprises at least one light source for illuminating the detection region of the test stick and at least one photodetector for detecting light emanating from the detection region.

79. The system of claim 78, wherein the light emanating from the detection region is within a predefined light emanation range.

80. The system of claim 79, wherein the inserter comprises an inserter region, configured such that when the inserter is inserted into the reading device, the inserter region aligns with the detection means, wherein the inserter region comprises at least one optical feature configured such that when the at least one light source illuminates the inserter region, the optical feature is such that light emanating from the inserter region falls outside the predefined light emanation range.

81. The system of claim 80, wherein the optical feature is a recess or hole.

82. The system of claim 80, wherein the optical feature provides the inserter region with a reflectance or transmittance different from that provided by the detection region.

83. The system of claim 79, wherein the predefined light emanation range is a predefined light intensity range.

84. The system of claim 73, wherein the reading device is at least one of an assay result reading device, an ovulation test device and a pregnancy test device.

85. The system of claim 73, wherein the reading device further comprises a wireless communication means, and wherein the wireless communication means is activated when the reading device operates in the second state.

86. The system of claim 85, wherein the inserter is not a test stick.

87. The system of claim 73, wherein the inserter is a used test stick, and wherein an error notification is activated when the reading device operates in the second state.

88. The system of claim 73, wherein the reading device is arranged to read a result of a first test when in the first state and a result of a second, different test when in the second state.

89. The system of claim 88, wherein the test stick is a first test stick and the inserter is a second test stick.

90. The system of claim 89, wherein the first test stick is suitable for measuring at least one analyte and the second test stick is suitable for measuring at least one different analyte.

91. The system of claim 89, wherein the first test stick is suitable for measuring at least one analyte at a first sensitivity, and the second test stick is suitable for measuring the at least one analyte at a second sensitivity.

92. A method of changing a state of a reading device, the reading device being arranged to operate in a first state for reading a result of a test performed using a test stick, the reading device comprising a detection means arranged to detect a first reading influenced by a detection region of the test stick, the method comprising:
inserting an inserter into the reading device; and
detecting, by the detection means, a second reading distinct from the first reading;
wherein the reading device operates in a second state in response to detecting the second reading.
